(19) United States
(12) Patent Application Publication
(10) Pub. No.: US 2017/0127985 A1
(43) Pub. Date: May 11, 2017

(54) SENSOR SET

(71) Applicant: MEDTRONIC MINIMED, INC., Northridge, CA (US)

(72) Inventors: Chase A. Thompson, Sherman Oaks, CA (US); Ashley N. Sullivan, Los Angeles, CA (US); David C. Antonio, Pasadena, CA (US); Jose J. Ruelas, San Fernando, CA (US); Eric Allan Larson, Simi Valley, CA (US)

(73) Assignee: MEDTRONIC MINIMED, INC.

(21) Appl. No.: 14/938,458

(22) Filed: Nov. 11, 2015

(51) Int. Cl. A61B 5/145; A61B 5/00; A61B 5/1477

(52) U.S. Cl. CPC A61B 5/14532; A61B 5/1477; A61B 5/0022

(57) ABSTRACT

An improved sensor set is provided for sensing of a body characteristic, such as glucose. The sensor set includes a mounting base for the sensor and a connector to connect to the mounting base. The connector may contain sensor electronics for wired or wireless communication to an external monitor or display. The mounting base includes a connector fitting adapted to fit into a tubular recess in the connector, wherein the connector fitting includes a key. In some embodiments, the proximal end of the sensor folds around the key such that there are contact pads on both sides of the key. The mounting base and connector include additional features, such as pegs and prongs that allow for unique fitting of compatible mounting bases and connectors, while locking out non-compatible components.

### BACKGROUND OF THE INVENTION

Field of the Invention

The present invention generally relates to a sensor set and insertion set for monitoring a body characteristic of the body, such as glucose. More particularly, the present invention relates to a sensor set including a mounting base and connector, where the connector and mounting base are configured to couple to each other.

Description of the Related Art

In recent years, a variety of electrochemical sensors have been developed for a range of applications, including medical applications for detecting and/or quantifying specific agents in a patient's blood and other body fluids. As one example, glucose sensors have been developed for use in obtaining an indication of blood glucose levels in a diabetic patient. Such readings can be especially useful in monitoring and/or adjusting a treatment regimen which typically includes regular administration of insulin to the patient. In this regard, blood glucose readings are particularly useful in conjunction with semi-automated medication infusion pumps of the external type, as generally described in U.S. Pat. Nos. 4,562,751; 4,678,408; and 4,685,903; or automated implantable medication infusion pumps, as generally described in U.S. Pat. No. 4,573,994.

Relatively small and flexible electrochemical sensors have been developed for subcutaneous placement of sensor electrodes in direct contact with patient blood or other extracellular fluid, wherein such sensors can be used to obtain periodic readings over an extended period of time. In one form, flexible transcutaneous sensors are constructed in accordance with thin film mask techniques wherein an elongated sensor includes thin film conductive elements encased between flexible insulative layers of polyimide sheet or similar material. Such thin film sensors typically include exposed electrodes at a distal end for subcutaneous placement in direct contact with patient blood or the like, and exposed conductive contact pads at an externally located proximal end for convenient electrical connection with a suitable monitoring device. Such thin film sensors hold significant promise in patient monitoring applications, but unfortunately have been difficult to place transcutaneously with the sensor electrodes in direct contact with patient blood or other body fluid. Improved thin film sensors and related insertion sets are described in commonly assigned U.S. Pat. Nos. 5,390,671; 5,391,250; 5,482,473; 5,299,571; 5,586,553 and 5,568,806, which are incorporated by reference herein.

Currently, there are sensor sets that include a mounting base, for placement on the patient's skin, which can be coupled to a connector with suitable sensor electronics (wired or wireless). Because the mounting base may be sold separately, it is possible to attach incompatible components together, which can compromise the sensor data. In addition, the structure of the current sensors allow for limited number of contact pads, and respective sensor electrodes.

### SUMMARY OF THE INVENTION

According to one embodiment, a sensor set for sensing a characteristic of a patient is provided, the sensor set comprising a sensor having at least two sensor electrodes thereon at a distal end for generating at least one electrical signal representative of a characteristic, such as blood glucose, of a patient, the sensor including at least two contact pads at a proximal end, wherein each of the at least two contact pads are conductively coupled to at least one of the at least two sensor electrodes; a mounting base operable for mounting onto a patient's skin, the mounting base including a connector fitting generally at a rear end of the mounting base, wherein the connector fitting includes a tubular element having a central bore formed therein for pass through reception of a portion of the sensor, a connector operable to couple to the mounting base, wherein the connector includes a tubular recess sized to receive the connector fitting of the mounting base and at least two connector contacts that are operable to be electrically coupled to the at least two contact pads of the sensor when the mounting base is coupled to the connector, wherein the connector fitting includes a key formed at one end, wherein the proximal end of the sensor folds around the key such that at least one of the at least two contact pads is on a first side of the key and at least one other of the at least two contact pads is on a second side of the key.

In further embodiments, the at least two connector contacts of the connector are compressible pins. In still further embodiments, the compressible pins compress into the first side of the key and the second side of the key when the mounting base is coupled to the connector.

In embodiments, the sensor includes a shorting path allowing for a shorted reference electrode and counter electrode. In alternate or further embodiments, the sensor includes a resistor.

The key may be substantially oval or rectangular in shape. In embodiments, the first side of the key is substantially flat. The second side of the key may also be substantially flat or it may include a substantially flat end portion and a step portion. One or both sides of the key may include seats or flats to receive the proximal end of the sensor. In embodiments, the key includes at least one prong adapted to fit into a prong recess formed in the mounting base. The first side of the key is substantially flat.

In embodiments, the mounting base includes at least one arm generally adjacent to the tubular element of the connector fitting, wherein the at least one arm is formed to fit into at least one corresponding arm recess formed in the connector when the connector is connected to the mounting base.

In embodiments, the connector fitting includes a first side rail formed on a first side of the connector fitting and a second side rail formed on a second side of the side rail, wherein the first side rail and second side rail are operable to slide into a first slot formed in the mounting base tubular recess and a second slot formed in the connector tubular recess when the connector is connected to the mounting base.

In embodiments, the connector includes sensor electronics, for example including a wireless transmitter operable to transmit signals from the mounting base. The at least two sensor electrodes may be operable to generate at least two electrical signals representative of the characteristic and the at least two connector contacts may be operable to receive the at least two electrical signals. These electrical signals may be transmitted via wireless transmitter or transmitted over wire.

In embodiments, the mounting base and the connector has releasably interengageable snap fit latch members operable to lock the mounting base to the connector.

### BRIEF DESCRIPTION OF THE DRAWINGS

For a more complete understanding of the present disclosure, reference is now made to the following figures, wherein like reference numbers refer to similar items throughout the figures:

FIG. 1 illustrates a perspective view of a connector and a mounting base.

FIG. 2 illustrates a perspective view of a connector and test plug.

FIG. 3 illustrates an expanded view of a connector with wireless sensor electronics.

FIG. 4 illustrates a perspective view of a sensor set with insertion tool.

FIG. 5 illustrates an expanded view of a mounting base, including sensor, and insertion tool.

FIG. 6 illustrates an expanded perspective view showing assembly of the mounting base components, including a sensor.

FIG. 7 illustrates an underside, expanded view of the mounting base, including sensor, shown in FIG. 6.

FIG. 8 illustrates an underside view of the mounting base shown in FIG. 6.

FIG. 9 illustrates another expanded view of a mounting base with retainer cap.

FIG. 10 illustrates another expanded view of a mounting base with retainer cap.

FIG. 11 illustrates an underside view of the retainer cap of FIG. 11.

FIG. 12 illustrates a perspective view of a mounting base with retainer cap.

FIG. 13 illustrates a perspective view of an insertion needle installed on an assembled mounting base and retainer cap.

FIG. 14 illustrates a sectional view taken generally on the line 11-11 of FIG. 10.

FIG. 15 illustrates a front end perspective view of a connector in the form of a cable connector.

FIG. 16 illustrates an exploded perspective view of a connector in the form of a cable connector.

FIG. 17 illustrates a perspective view showing sliding removal of the insertion needle from a sensor set, following placement of the mounting base onto the skin of a patient.

FIG. 18 illustrates a perspective view showing a sensor set and cable connector mounted onto the skin of a patient, following removal of the insertion needle.

FIG. 19 illustrates a top view of a test plug.

FIG. 20 illustrates a side view of a test plug.

FIG. 21 illustrates a perspective view of a test plug.

FIG. 22 illustrates a partial perspective view of a connector.

FIG. 23 illustrates a perspective view of a test plug.

FIG. 24 illustrates a partial perspective view of a mounting base or test plug.

FIGS. 25A-C illustrate sectional views taken along a socket fitting of a mounting base.

FIGS. 25D-E illustrate perspective, expanded views of pins used as contacts in a mounting base.

FIG. 25F illustrates a cut away view of a mounting base connected to a test plug.

FIG. 26 illustrates a perspective view of a mock-up showing the connection between a test plug or connector and a mounting base.

FIG. 27A-27F illustrates cut away views of various lock-out connections between test plug/connector and a mounting base.

FIG. 28 illustrates a diagram of contact pads for a sensor.

### DETAILED DESCRIPTION

The following description and the drawings illustrate specific embodiments sufficiently to enable those skilled in the art to practice the system and method described. Other embodiments may incorporate structural, logical, process and other changes. Examples merely typify possible variations. Individual elements and functions are generally optional unless explicitly required, and the sequence of operations may vary. Portions and features of some embodiments may be included in, or substituted for, those of others.

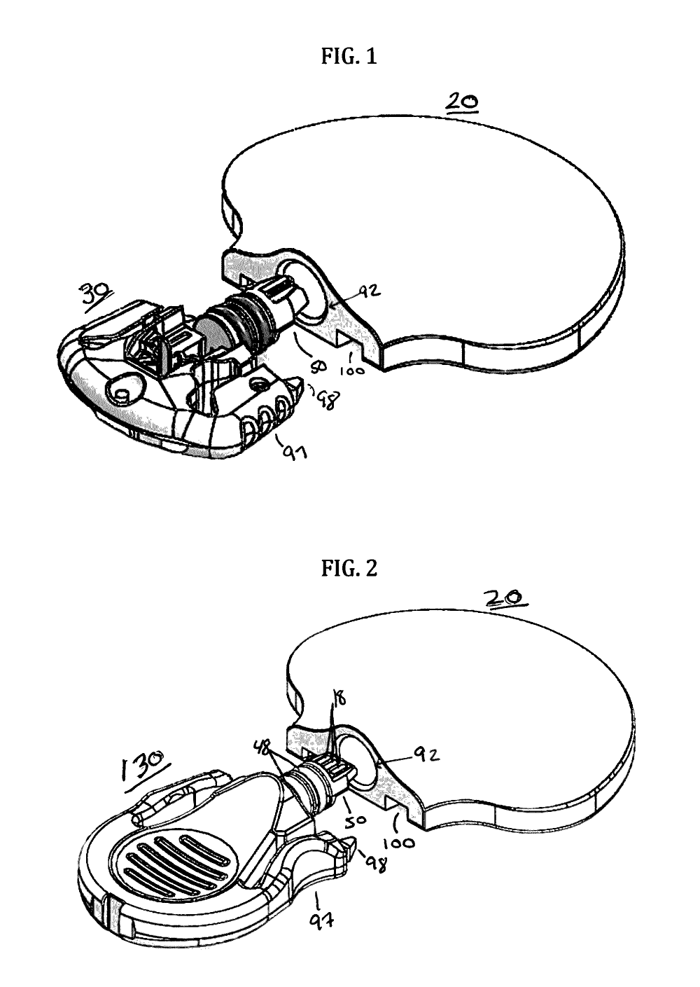

As shown in the exemplary drawings, an improved sensor set is provided for monitoring a body characteristic of the body. One example body characteristic is the blood glucose level of the body. As shown in FIG. 1, an embodiment of a sensor set includes a sensor mounting base 30 and connector 20. The connector shown in FIG. 1 includes wireless sensor electronics that have a sensor transmitter (not shown) inside the housing. However, as shown in embodiments in other figures, the connector may be wired directly to a monitor, which may or may not include a display. It may include only a wire connector or may include some sensor electronics that also include a wire connector, depending on the needs of the user. The mounting base 30 includes latch arms 97 and latch tips 98, which can interact with latch recesses 100 on the connector 20. When the mounting base 30 is coupled to the connector 20, the latch tips 98 lock into the latch recesses 100. They can be released by pressing on the latch arms 97 so that the connector 20 can be removed.

The connector 20 may also be used with a test plug 130, one embodiment of which is shown, in FIG. 2. Test plug 130 has the same general configuration with respect to the connector 20 connections as does the mounting base 30. When attached to the sensor connector 20, the test plug provides a tight seal, allowing a patient to wash those components. This is particularly useful in the embodiments with wireless transmitters, especially those that are sealed within the connector 20. The test plug 130 also allows for testing of wireless transmitters or other sensor electronics in the connector 20 without receiving data from any sensor. Thus sensor electronics can be tested alone and without external sensor noise.

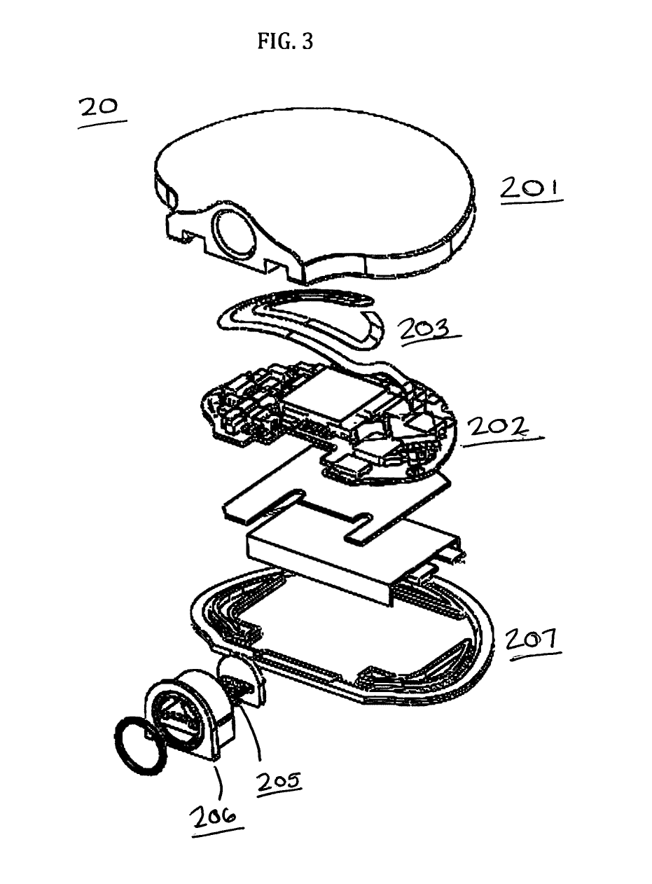

An example connector 20 is shown in FIG. 3, which includes sensor electronics with a wireless transmitter. The housing 201 and 207 is formed over a circuit board 202 with antenna 203. The contact plug/pin housing 204 holds the molded pins 205 and sits inside the housing 201/207 such that when the sensor mounting base 30 is connected to the sensor 20, the sensor electronics can receive signals from the sensor housed in the mounting base 30. Sensor electronics including wireless transmitters are discussed, for example, in U.S. Pat. No. 7,602,310, which is herein incorporated by reference.

In embodiments of the present invention, the sensor set and any related monitor are for determining glucose levels in the body and/or body fluids of the user and may be used in conjunction with automated or semi-automated medication infusion pumps of the external or implantable types as described in U.S. Pat. Nos. 4,562,751, 4,678,408, 4,685,903, and 4,573,994, which are herein incorporated by reference, to deliver insulin to a diabetic patient. However, it will be recognized that further embodiments of the invention may be used to determine the levels of other agents, characteristics or compositions, such as hormones, cholesterol, medication concentrations, pH, oxygen saturation, viral loads (e.g., HIV), or the like. In other embodiments, the sensor set may also include the capability to be programmed or calibrated using data received by the sensor electronics, or may be calibrated at the monitor device. The sensor system is primarily adapted for use in subcutaneous human tissue. However, still further embodiments may be placed in other types of tissue, such as muscle, lymph, organ tissue, veins, arteries or the like, and used in animal tissue. It will be understood that the term “patient” can be broadly construed to encompass humans and other animals, and that the term “blood” encompasses patient blood and other extracellular patient fluids. Embodiments may provide sensor readings on an intermittent or continuous basis.

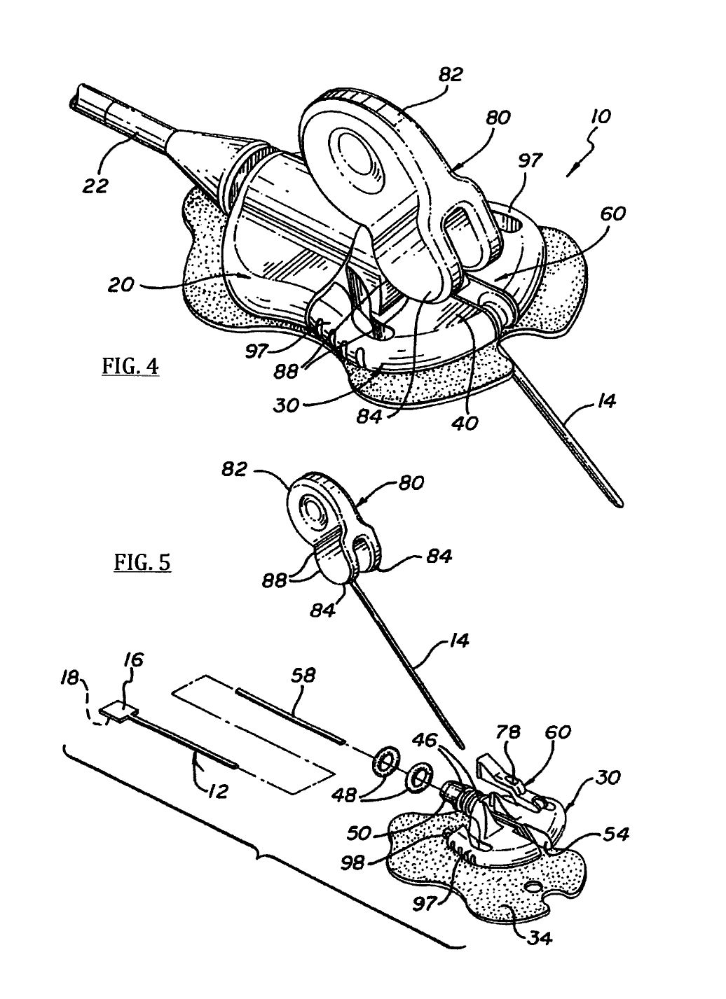

The sensor set uses an electrode-type sensor, as described in more detail below. However, in alternative embodiments, the system may use other types of sensors, such as chemical based, optical based or the like. In further embodiments, the sensors may be of a type that is used on the external surface of the skin or placed below the skin layer or the user. Certain embodiments of a surface mounted sensor would utilize the interstitial fluid harvested from underneath the skin.

Where the connector has sensor electronics including a wireless transmitter, the sensor electronics generally include the capability to transmit data. However, in alternative embodiments, these sensor electronics may include a receiver or the transmitter may be a transceiver with the capability to receive data. FIGS. 4-18 show embodiments without sensor electronics other than a direct connection to a wire that can carry signals to separate sensor electronics, which may include a monitor or display and may also (or alternatively) transmit data to an external monitor or display.

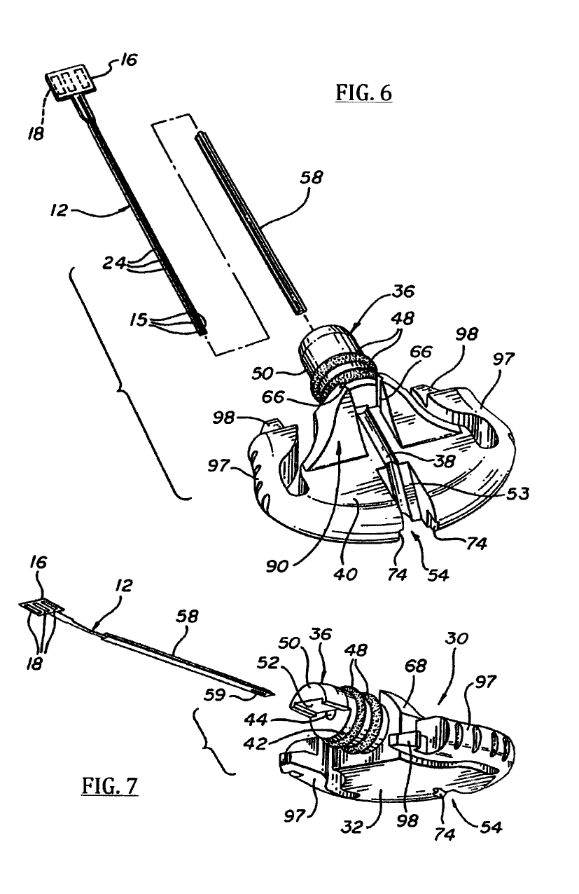

As shown in FIGS. 6-8, the flexible thin sensor 12 comprises a relatively thin and elongated element which can be construed according to so-called thin mask techniques to include elongated conductive elements 24 (FIG. 6) embedded or encased between layers of a selected insulative sheet material such as polyimide film or sheet. The proximal end or head 16 of the sensor 12 is relatively enlarged and defines the conductive contact pads 18, which are exposed through the insulative sheet material for electrical connection to the cable 22, as will be described in more detail. An opposite or distal segment of the sensor 12 includes the corresponding plurality of exposed sensor electrodes 15 for contacting patient body fluid when the sensor distal segment is placed into the body of the patient. The sensor electrodes 15 generate electrical signals representative of patient condition, generate electrical signals representative of patient condition, wherein these signals are transmitted via the contact pads 18 and connector, which may include sensor electronics (wired or wireless) or just a connection to a wire, to an appropriate monitoring device (not shown) for recordation and/or display to monitor patient condition. Further description of flexible thin film sensors of this general type may be found in U.S. Pat. No. 5,391,250, which is herein incorporated by reference. Further description of wired sensor electronics may be found in U.S. Pat. No. 7,602,310, which is herein incorporated by reference.

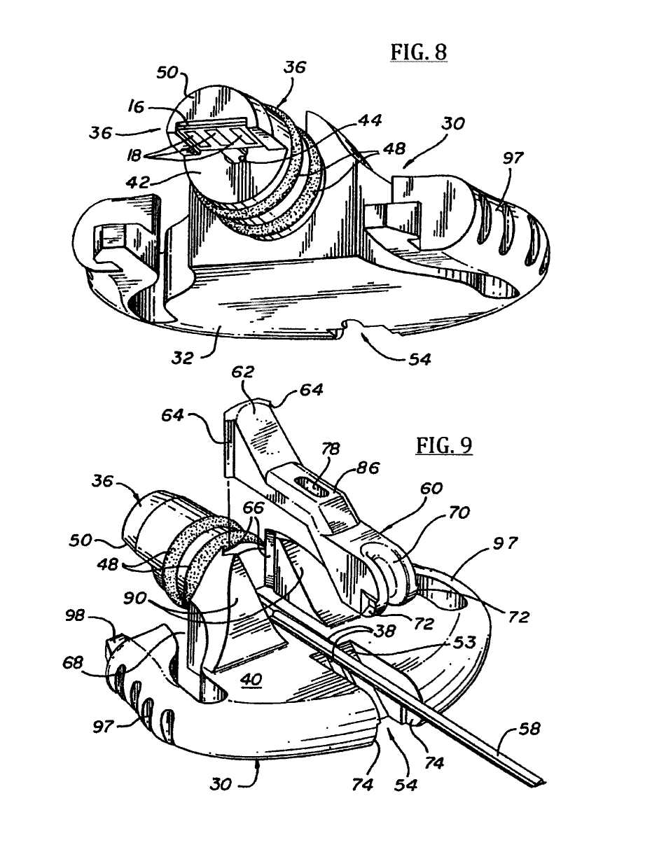

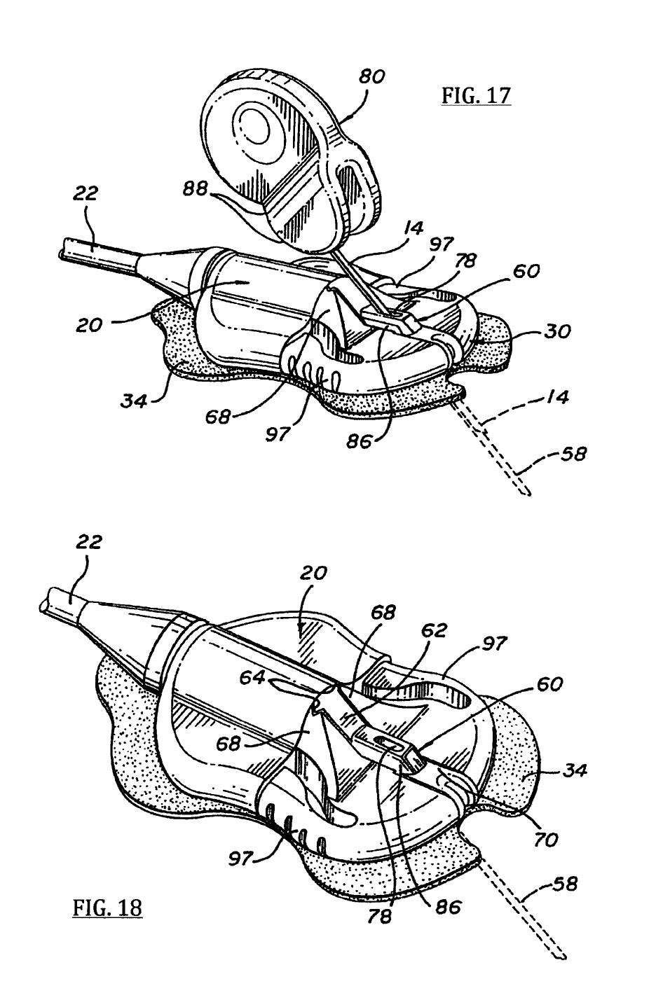

The sensor 12 is carried by the sensor set, specifically on the mounting base 30, which is adapted for placement onto the skin of a patient (FIGS. 17-18) at the selected insertion site. The sensor set generally comprises a compact mounting base 30 having a generally planar or flat underside surface 32 (FIGS. 7-8) attached to an adhesive patch 34 for press-on adhesive mounting onto the patient's skin. It is understood that alternative methods for attaching the mounting base to the skin of a patient also may be contemplated. The mounting base 30 is generally constructed out of lightweight plastic so that it may be comfortably worn throughout numerous activities by a patient. The mounting base 30 may be constructed as a unitary molding of lightweight plastic to include a connector fitting 36 for slide-fit coupling with the sensor connector 20, which may include a wireless transmitter, wired electronics, or merely a cable for connection to external sensor electronics and/or monitor. The connector fitting 36 cooperates with an upwardly open recessed groove or channel 38 formed in an upper surface 40 of the mounting base 30 (e.g., FIG. 5) to receive and support the sensor 12.

FIGS. 5-8 show a mounting base 30 to comprise a rearwardly projecting tubular element 42 defining a central bore 44 aligned generally coaxially with a rearward end of the recessed channel 38. The tubular element 42 includes external grooves 46 (FIG. 5) for receiving seal rings 48 adapted for sealed slide-fit engagement with the cable connector 20, as will be described. The rearward end of the tubular element 42 terminates in a narrow, generally oval or rectangular shaped fitting key 50 formed as a rearward extension thereof.

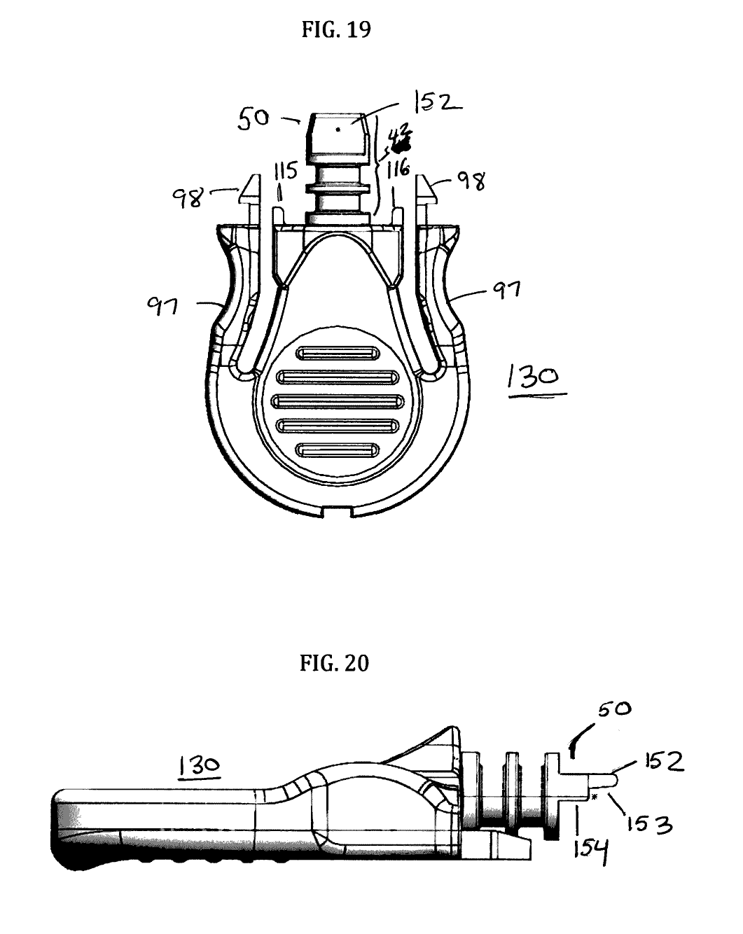

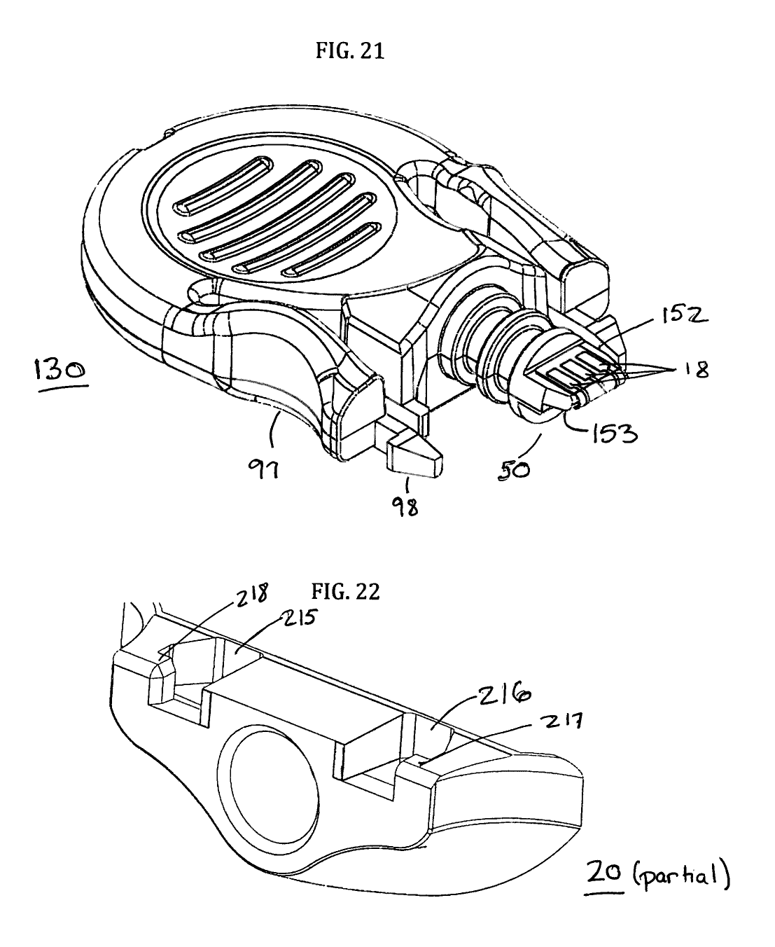

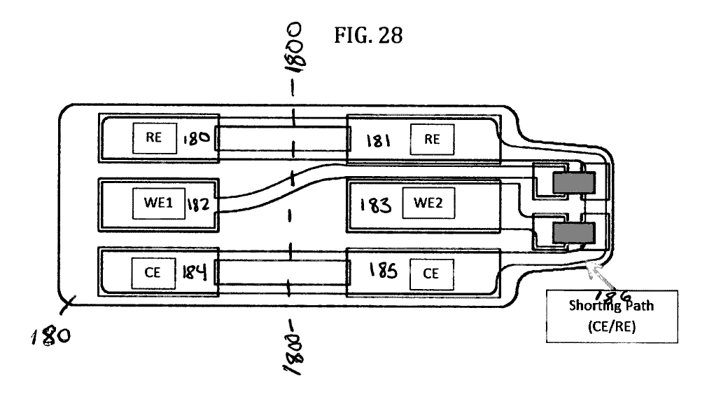

As shown in FIGS. 19-21, the top and the bottom of the key 50 are each substantially flat and each incorporate shallow flats or seats 152 and 153. The recessed seats are sized and shaped to receive and support the proximal head 16 of the sensor 12, with the proximal end of the elongated sensor 12 extending from said head 16 through the bore 44 and lying within the recessed channel 38. Moreover, the recessed seats may include a shelf 154 (FIG. 20) that allows the bottom of the key to step from a narrow depth to a slightly wider depth. This shelf allows for the unique fitting of current keys into only compatible connecting pieces on sensor connectors, which include matching recesses. The shelf further improves tolerance and robustness of the connections. As can be seen in FIG. 21, the proximal head 16 of the sensor 16 may be configured such that it folds around the key 50 so that the sensor contacts are on both seats 152 and 153. In embodiments of this configuration, the proximal head 16 of the sensor includes 2 sets of contact pads 18. For example, in FIG. 28 one embodiment of a schematic for the contact pads is shown. The contact pads shown are two for a reference electrode (180, 181), two for a counter electrode (184, 185), and one for each of two working electrodes (182, 183). A shorting path 186 allows for a shorted reference electrode/counter electrodes and a resistor may be used to account for additional working electrode(s) and counter electrode(s). By allowing for additional contact pads in the same space, additional electrodes may be used in the sensor without requiring additional size of the mounting base or connector. In the configuration showed in FIG. 28, the fold of the substrate 180 is generally along the line 1800-1800 so that one set of contact pads is on each side of the key 50. In alternative embodiments a single set of contacts could be included on just one side of the key 50, as shown in other embodiments herein.

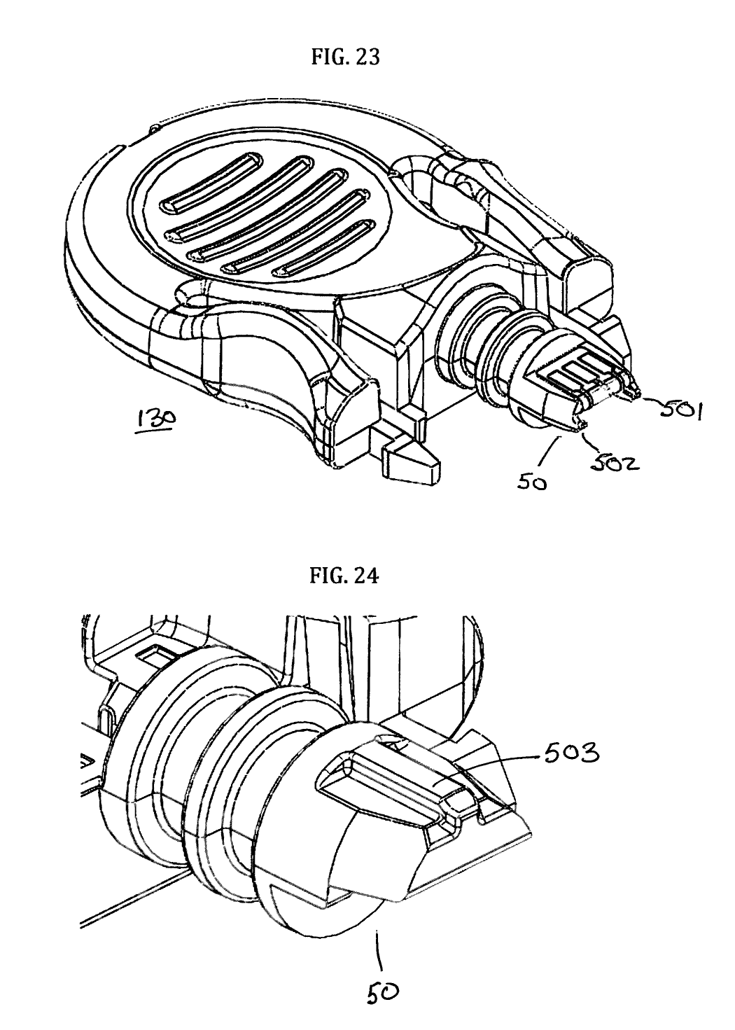

In still further embodiments, as shown in FIG. 23, the key 50 includes prongs 501 and 502 that fit into respective prong recesses in the connector (not shown) and lockout other non-compatible connectors. The prongs also help assist in rotational stability of the coupling and assist in ensuring a good connection between the connector and mounting base.

In alternate embodiments, shown for example in FIG. 8, the rearward end of the tubular element 42 terminates in a generally D-shaped or half-circle fitting key 50 formed as a rearward extension of the tubular element and which incorporates a shallow recessed flat or seat 52 formed at the rearward end of the bore 44. The recessed seat 52 receives and supports the proximal head 16 of the sensor 12.

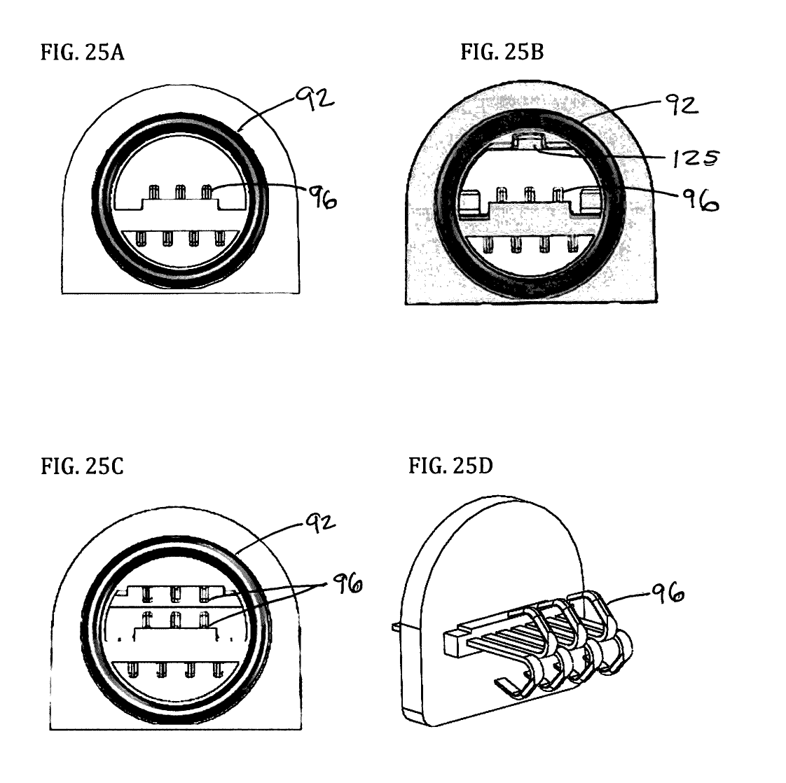

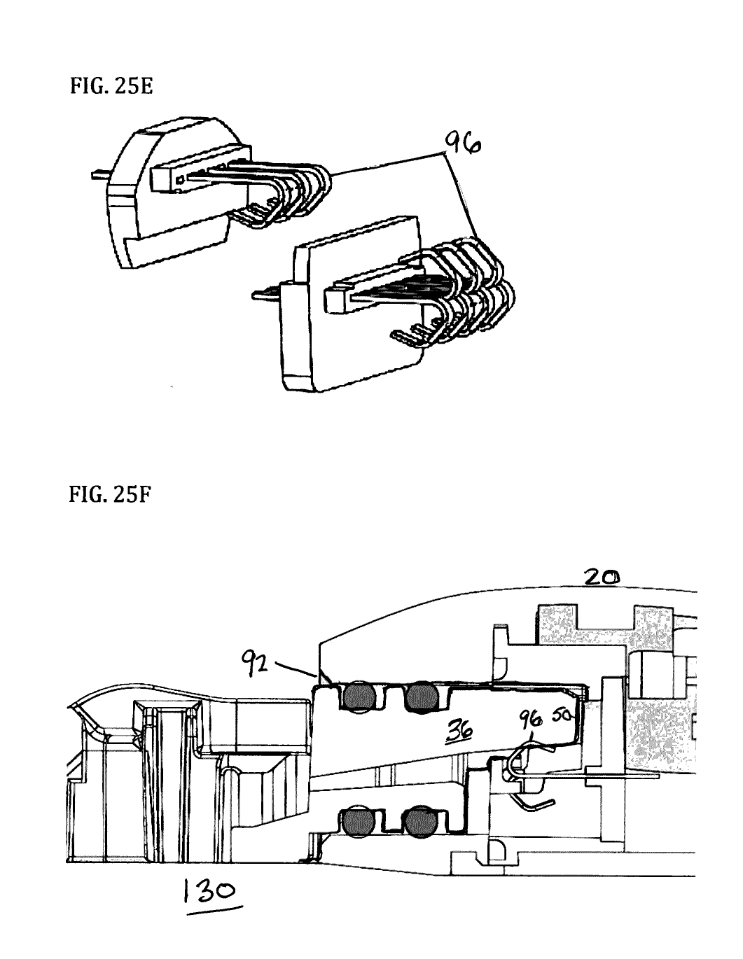

In still further embodiments, the rearward end of the tubular element 42 terminates in a generally D-shaped or half-circle fitting key 50 that has been cut out on top to form a rail 503 (as shown in FIG. 24). This rail 503 corresponds to a trench 125 (as shown in FIG. 25B) in the mounting base. The rail provides stability and prevents other non-compatible components, which do not include the rail/trench configuration, from coupling with components that have the rail/trench configuration.

In embodiments, the head 16 of the sensor 12 is secured and seated within the recessed seat(s) 52 or 152, 153 by means of a suitable adhesive or the like. In addition, subsequent to placement of the sensor 12 through the bore 44, the bore 44 may be hermetically sealed with suitable sealant such as curable silicone sealer or the like. For facilitated slide-fit engagement with the sensor connector 20, the seat 52 may be formed to ramp angularly rearwardly and upwardly from a central axis of the bore 44, thereby supporting the sensor head 16 with the contact pads 18 presented downwardly and angularly rearwardly.

Because it is possible for both configurations of keys discussed above to exist in various mounting bases of differing compatibilities, the key further serves to prevent use of one configuration of mounting base 30 with the other configuration of connector 20. Thus, the key allows to ensure compatibility of sensors to connectors and to related monitors and prevents chemically or technically incompatible parts from being used with each other.

In embodiments, the recessed channel 38 in the mounting base 30 thus receives and supports the proximal segment of the thin film sensor 12. As shown in FIGS. 6, 9 and 10, the recessed channel 38 extends forwardly from the fitting bore 44 with a generally horizontal orientation, and then turns downwardly and forwardly at an angle of about 45 degrees to extend along an angled face 53 within a forwardly open gap 54 formed in the front end or nose of the mounting base. In some embodiments, a cannula 58 is slidably fitted over at least a portion of the proximal segment of the sensor 12, to extend also over the distal segment to encase and protect the sensor. In the preferred form, the cannula is constructed from a lightweight plastic material such as a urethane based plastic, and has a double lumen configuration. The double lumen cannula 58 is especially suited for slide-fit engagement with and disengagement from the insertion needle 14, as will be describe din more detail, and includes a window 59 to expose the sensor electrodes 15. The specific cannula construction for receiving and supporting the sensor 12, and for slidably interfitting with the insertion needle 14, is shown and described in more detail in U.S. Pat. No. 5,586,553, which is herein incorporated by reference.

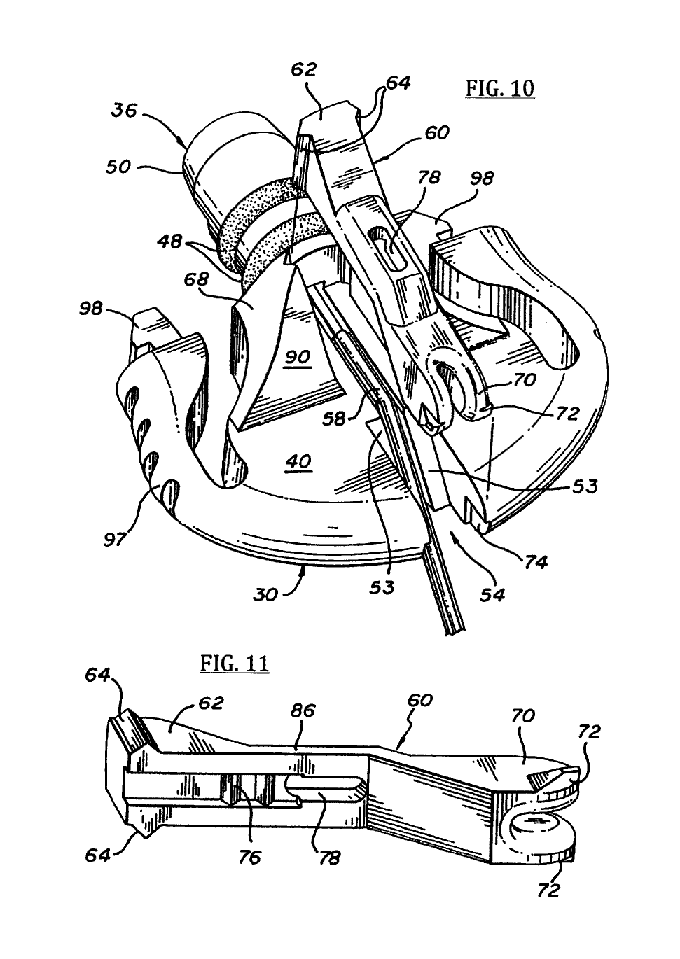

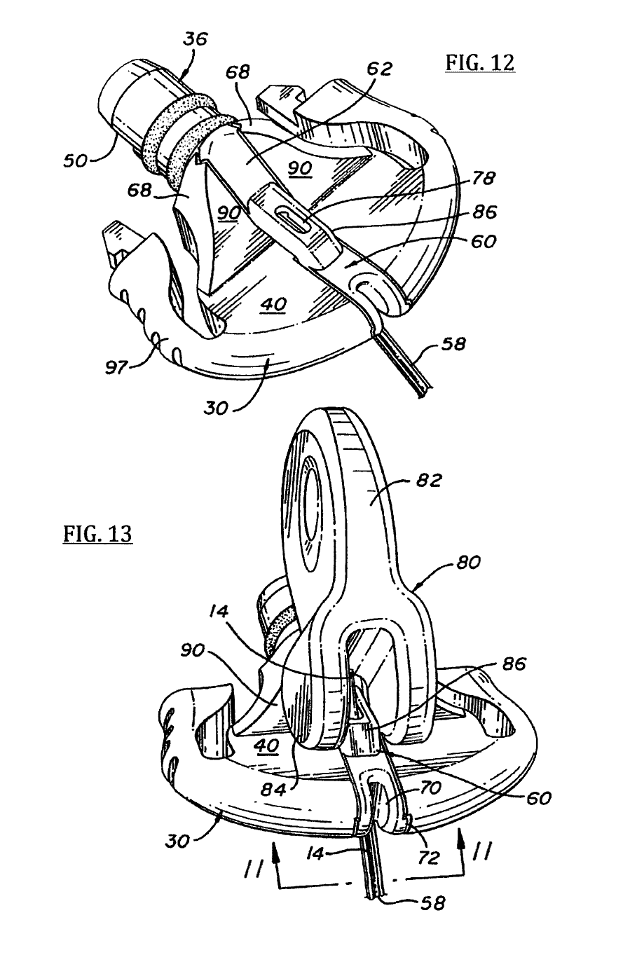

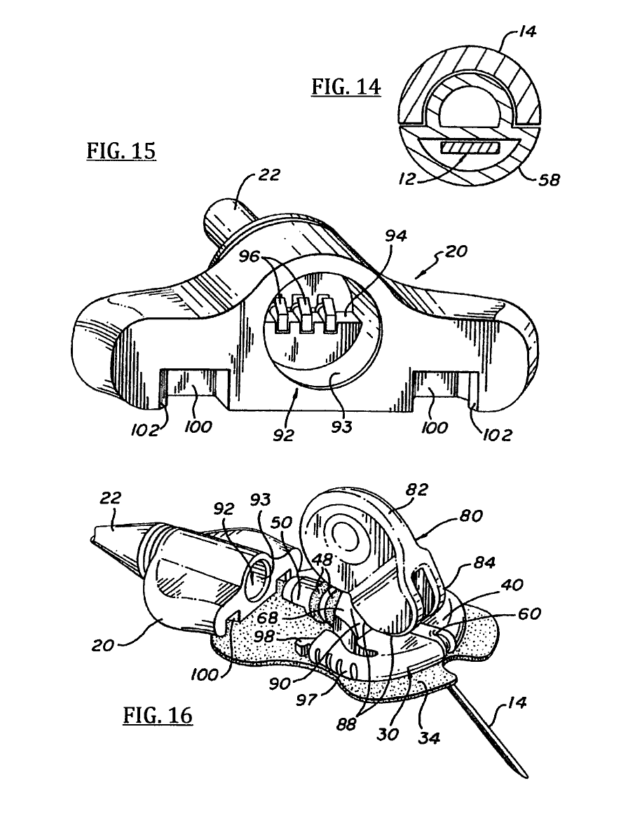

In further embodiments, an insertion method is included as part of the sensor set. For example, a retainer cap 60 allows for insertion of the sensor set. In embodiments, the proximal end of the sensor 12 and the portion of the cannula 58 thereon are folded as shown in FIG. 10 to follow the contour of the mounting base channel 38, so that the distal segment of the sensor and the cannula thereon extend and protrude downwardly and forwardly from the front of the mounting base 30. The sensor and cannula may be captured and retained in this orientation by a retainer cap 60 shown in FIGS. 10-12. This retainer cap 60 also may be formed conveniently and economically as a lightweight plastic molding and includes means for quick and easy snap fit installation onto the mounting base 30. When the retainer cap 60 is assembled with the mounting base 30, these components cooperatively close the top of the channel 38 to capture and retain the sensor and cannula therein. The retainer cap 60 further defines a needle port 78 (FIGS. 12 and 13) for pass through reception of the insertion needle 14. Them insertion needle 14 has a hollow and longitudinally slotted configuration (FIG. 14) with a pointed or sharpened tip and a rear end anchored to an enlarged hub 80. The hub 80 is manually manipulated to fit the needle 14 through the cap port 78, in order to slide the slotted needle into engagement with the cannula 58 within the forwardly and downwardly angled portion of the channel 38. In this regard, the needle port 78 is sized and shaped to orient the insertion needle 14 for proper angular and rotational alignment with the cannula 58 to ensure correct slide-fit engagement therebetween.

More particularly, the hub 80 includes an enlarged tab-like wing 82 adapted for easy grasping and handling between the thumb and index finger. This enlarged wing 82 projects upwardly from a bifurcated nose 84 which is sized and shaped to seat onto the mounting base upper surface 40, on opposite sides of a raised central section 86 of the retainer cap 60. The hub nose 84 is contoured to defined keyed alignment or guide surfaces 88 for matingly contacting associated keyed alignment surfaces on the mounting base 30, defined by the upper surface 40 and an angularly presented forward face 90 of the support brackets 68. With this geometry, the hub 80 is slidably displaced against the mounting base 30 with the insertion needle 14 extending into and through the cap port 78 at the correct angular and rotational orientation for slide-fit engagement with and disengagement from the cannula 58. In the preferred form, the insertion needle 14 slidably assembles with the cannula 58 as described in U.S. Pat. No. 5,586,553, which is herein incorporated by reference, to provide a generally circular cross sectional profile (FIG. 14) protruding from the mounting base. The insertion set discussed herein is more particularly described in U.S. Pat. No. 7,660,615, which is herein incorporated by reference.

FIGS. 13-15 show the connector 20 for coupling with the assembled insertion set 10. A cable connector is used for wired connection to a monitor or display. The connecting components of the cable connector can instead be on a connector including sensor electronics that allow wired or wireless transmission to a monitor or display. As shown, the cable connector 20 comprises a compact coupling element which can also be constructed from lightweight molded plastic. The cable connector 20 defines a socket fitting 92 for mating slide-fit engagement with the rear cable fitting 36 of the mounting base 30. This socket fitting 92 has a cylindrical entry portion 93 which merges with a generally D-shaped or half-circle step portion 94 sized to receive the D-shaped key 50 of the fitting 36. As shown, the socket fitting 92 includes a plurality of conductive contacts 96 (FIG. 15) positioned on the step portion 94 for electrically coupled engagement with the contact pads 18 on the sensor 12, when the insertion set 10 and cable connector 20 are coupled together as viewed in FIG. 16. When assembled, the seal rings 48 sealingly engage the entry portion 93 of the socket fitting 92 to provide a sealed connection between the components. The D-shaped geometry of the interfitting components 50 and 94 ensure one-way interconnection for correct conductive coupling of the cable 22 to the sensor 12.

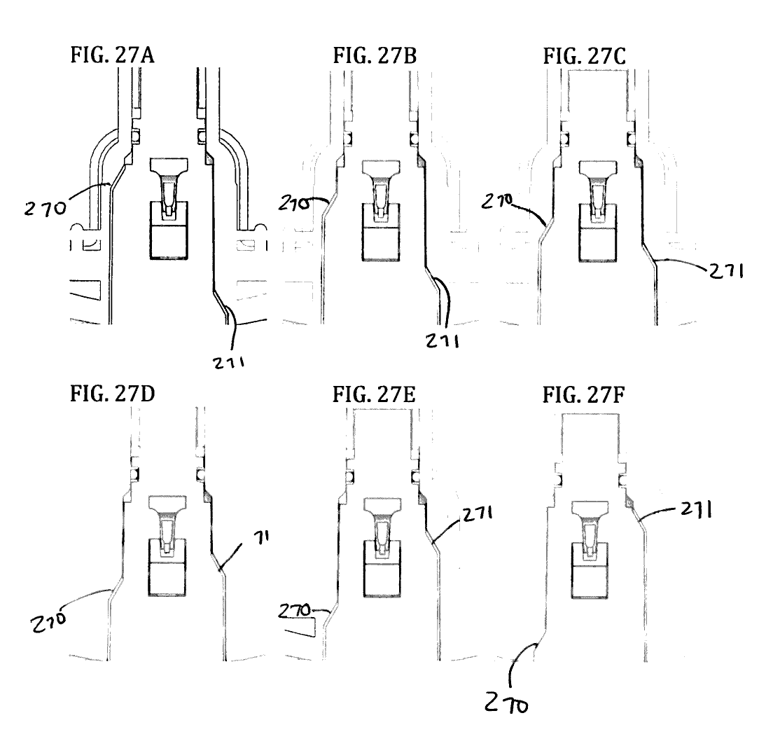

Several embodiments of the conductive contacts 96 are shown in FIG. 27A-F. While certain numbers of conductive contacts are shown in these figures, and corresponding numbers of sensor contact pads are shown in other figures herein, it is contemplated that different numbers of conductive contacts and corresponding sensor contact pads could be used as long as they are sufficient to determine data related to the sensed body characteristic. FIG. 27A-C show cut-away views of the socket fitting 92 with the interior conductive contacts 96. FIG. 27A shows the fitting of a generally D-shaped portion 94 with the conductive contacts 96 being pins that touch the contact pads of the key 50 when the key is fit into the socket fitting 92. FIG. 27B shows another embodiment where the D-shape has been slightly cut away and a railing added that fits into the runner 125. The runner 125 allows for both blocking the non-compatible components from being used with this sensor connector but also gives additional stability, such as rotational stability. FIG. 27C shows an embodiment where the conductive contacts are on both sides of the key, so that there are 2 sets of pins 96 that will contact the conductive contacts. Further views of the conductive pins are shown in FIG. 27D (pins from FIGS. 27A and 27B) and in FIG. 27E (pins from FIG. 27C, shown in expanded form). FIG. 27F shows an embodiment of a test plug 130 fitting into a connector 20. The form of the pins allows for some compression of the pins when the key is inserted into the socket. Unlike in a situation with 2 non-flexible contacts that need exact molding so that they touch each other, this compression gives assurance that the contact pads will contact the pins every time. In addition, the pins will provide additional stability while they push into the contact pads of the key 50.

The mounting base 30 and the cable connector 20 are retained in releasably coupled relation by interengaging snap fit latch members. As shown, the mounting base 30 is formed to include a pair of rearwardly projecting cantilevered latch arms 97 which terminate at the rearward ends thereof in respective undercut latch tips 98. The latch arms 97 are sufficiently and naturally resilient for movement relative to the remainder of the mounting base 30, to permit the latch arms to be squeezed inwardly toward each other. The permissible range of motion accommodates snap fit engagement of the latch tips 98 into a corresponding pair of latch recesses 100 formed in the cable connector 20 on opposite sides of the socket fitting 92, wherein the latch recesses 100 are lined with latch keepers 102 for engaging said latch tips 98. The components can be disengaged for uncoupling when desired by manually squeezing the latch arms 97 inwardly toward each other for release from the latch keepers 102, while axially separating the mounting base 30 from the cable connector 20.

Further as shown in FIG. 19 the test plug 130 or connector 30 (not shown in FIG. 19) includes anti-rotation arms 115 and 116. In certain embodiments, as in FIG. 19, the anti-rotation arms are generally adjacent to the tubular element 42, which includes the key 50. The mounting base 20 (partially shown in FIG. 22) includes anti-rotation arm recesses 215 and 216, which correspond and fit with the anti-rotation arms. In the embodiment shown in FIG. 22, the mounting base also includes catches 217 and 218, which can catch anti-rotation arms if they are formed with hooks, like the latch tips 98 of the test plug/connector. These anti-rotation components help to prevent rotation of the mounting base and test plug/connector with respect to each other and also help to ensure that the mounting base is only used with compatible test plugs/sensor connectors, avoiding compatibility issues of physical or technical nature. In further embodiments, the width and/or height of the anti-rotation arms may be varied to lock-out previous non-compatible components.

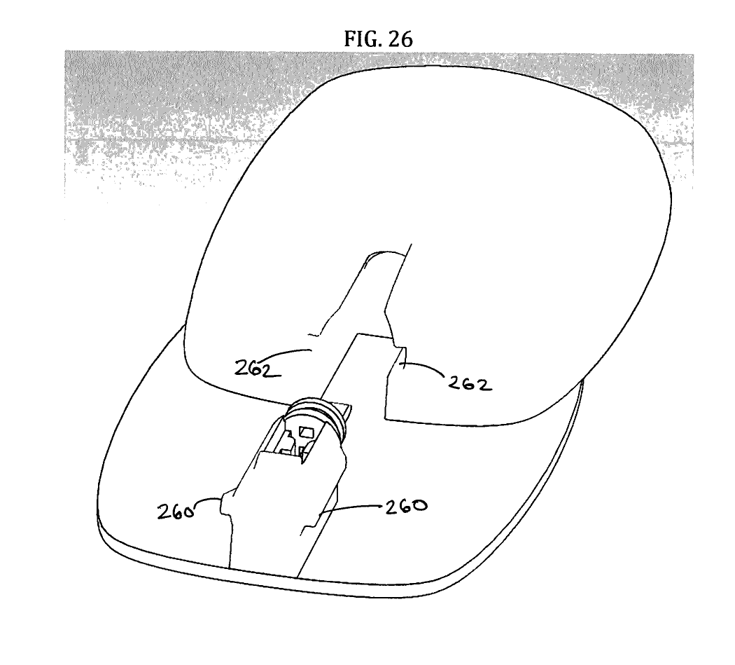

Additional embodiments are contemplated to provide stability and/or lockout from incompatible components. For example, in FIG. 26, a mock-up of the connecting portions of a mounting base and connector are shown, where the mounting base includes side rails 260 that fit into slots 262 in the connector. FIGS. 27A-27F show various examples of combinations of side rail lengths that could be used to lock out different versions of the mounting base and connector. As can be seen, the lengths of the rails are set up to match the lengths of the slots so that a mounting base with the incorrect rail lengths will not fit into a connector.

Any of the particular features discussed herein that may be used to lock-out non-compatible components may be used alone or in combination with each other, creating many possible iterations of connection configurations. Thus, it will be possible to ensure that many variations of components that are not compatible will not be able to connect to each other.

The sensor set of the present invention is mounted on the patient's skin quickly and easily to transcutaneously place the sensor 12. In one method, the mounting base 20 and connector 20 are initially coupled together by engaging the snap fit latch members. The hub 80 is also initially attached. The set is then pressed against the patient's skin, typically after removing a protective needle guard (not shown) and a release film (also not shown) from the underside of the adhesive patch 34 to expose a pressure sensitive adhesive thereon. Pressing the set against the skin causes the insertion needle 14 to pierce the skin and thereby carry the cannula 58 with the sensor electrodes 15 thereon to the desired subcutaneous position. The insertion needle 14 is then slidably disengaged from the cannula and sensor by withdrawing the needle from the patient. The insertion set 10 can be affixed more securely to the patient, if desired, by an overdressing (not shown). Alternatively, the mounting base may be affixed to the patient's skin before connecting to the connector. Thus, the connector would be connected to the mounting base after the mounting base is comfortably attached to the skin of the patient. It is also possible to disconnect the connector when the patient wishes to shower or wash the components. At this point, the test plug can be connected to the mounting base to protect the electronic components inside the sensor.

When it is necessary or desirable to remove the sensor from the patient, the insertion set is simply removed from the patient's skin to withdraw the sensor from the subcutaneous site. The insertion set 10 is quickly and easily disassembled from the cable connector 20 by appropriate release of the snap fit latch members. A new insertion set 10 can then assembled with the cable connector and quickly placed on the patient to subcutaneously position a new sensor.

The foregoing description of specific embodiments reveals the general nature of the disclosure sufficiently that others can, by applying current knowledge, readily modify and/or adapt the system and method for various applications without departing from the general concept. Therefore, such adaptations and modifications are within the meaning and range of equivalents of the disclosed embodiments. The phraseology or terminology employed herein is for the purpose of description and not of limitation.

What is claimed is:

1. A sensor set for sensing a characteristic of a patient, the sensor set comprising:
a sensor having at least two sensor electrodes thereon at a distal end for generating at least one electrical signal representative of a characteristic of a patient, the sensor including at least two contact pads at a proximal end, wherein each of the at least two contact pads are conductively coupled to at least one of the at least two sensor electrodes;
a mounting base operable for mounting onto a patient's skin, the mounting base including a connector fitting generally at a rear end of the mounting base, wherein the connector fitting includes a tubular element having a central bore formed therein for pass through reception of a portion of the sensor,
a connector operable to couple to the mounting base, wherein the connector includes a tubular recess sized to receive the connector fitting of the mounting base and at least two connector contacts that are operable to be electrically coupled to the at least two contact pads of the sensor when the mounting base is coupled to the connector,
wherein the connector fitting includes a key formed at one end, wherein the proximal end of the sensor folds around the key such that at least one of the at least two contact pads is on a first side of the key and at least one other of the at least two contact pads is on a second side of the key.

2. The sensor set of claim 1, wherein the at least two connector contacts of the connector are compressible pins.

3. The sensor set of claim 2, wherein the compressible pins compress into the first side of the key and the second side of the key when the mounting base is coupled to the connector.

4. The sensor set of claim 1, wherein the sensor includes a shorting path allowing for a shorted reference electrode and counter electrode.

5. The sensor set of claim 1, wherein the sensor includes a resistor.

6. The sensor set of claim 1, wherein the key includes at least one prong adapted to fit into a prong recess formed in the mounting base.

7. The sensor set of claim 1, wherein the first side of the key is substantially flat.

8. The sensor set of claim 7 wherein the second side of the key includes a substantially flat end portion and a shelf portion.

9. The sensor set of claim 1, wherein the first side of the key and the second side of the key each include seats to receive the proximal end of the sensor.

10. The sensor set of claim 1, wherein the mounting base includes at least one arm generally adjacent to the tubular element of the connector fitting, wherein the at least one arm is formed to fit into at least one corresponding arm recess formed in the connector when the connector is connected to the mounting base.

11. The sensor set of claim 1, wherein the connector fitting includes a first side rail formed on a first side of the connector fitting and a second side rail formed on a second side of the side rail, wherein the first side rail and second side rail are operable to slide into a first slot formed in the mounting base tubular recess and a second slot formed in the connector tubular recess when the connector is connected to the mounting base.

12. The sensor set of claim 1, wherein the connector includes a wireless transmitter operable to transmit signals from the mounting base.

13. The sensor set of claim 1, wherein the characteristic is blood glucose.

14. The sensor set of claim 1, wherein the at least two sensor electrodes are operable to generate at least two electrical signals representative of the characteristic and the at least two connector contacts are operable to receive the at least two electrical signals.

15. The sensor set of claim 1, wherein the mounting base and the connector have releasably interengageable snap fit latch members operable to lock the mounting base to the connector.
